(19) United States
(12) Patent Application Publication
(10) Pub. No.: US 2020/0138351 A1
(43) Pub. Date: May 7, 2020

(54) Method for Measuring Fluorescence in Ocular Tissue

(71) Applicant: Cognoptix, Inc., Concord, MA (US)

(72) Inventors: Charles Kerbage, Arlington, MA (US); Paul D. Hartung, Acton, MA (US)

(21) Appl. No.: 16/430,145

(22) Filed: Jun. 3, 2019

Related U.S. Application Data
(63) Continuation of application No. 15/035,549, filed on May 10, 2016, now abandoned.

(51) Int. Cl. A61B 5/1455; A61B 5/00; A61B 3/117; A61K 47/60; A61B 3/14; A61B 5/145; A61K 49/00

(52) U.S. Cl. CPC A61B 5/14555; A61B 5/4088; A61B 3/1173; A61B 5/0071; A61K 49/0021; A61K 47/60; A61B 3/14; A61B 5/14546; A61B 5/4082; A61B 5/0082

(57) ABSTRACT

A method is provided for ophthalmic measurements, wherein the amount of a fluorophore is detected in ocular tissue and the obtained fluorescence signals are normalized by performing a ratio.

### RELATED APPLICATIONS

This application is a continuation of U.S. application Ser. No. 15/035,549, which is the U.S. National Stage Application of International Application No. PCT/US2013/069598 filed on Nov. 12, 2013, published in English. The entire teachings of the above applications are incorporated herein by reference.

### FIELD OF THE INVENTION

The present invention relates to the field of ophthalmic measurements, wherein the amount of a fluorophore is detected in ocular tissue.

### BACKGROUND OF THE INVENTION

It is always desirable to detect diseases early in their progress. Early detection enables early treatment which has generally been proven to yield a higher success rate in treating various diseases. It has been discovered that analyzing peoples' eyes, and in particular the lenses of the eyes, can yield indications of various types of diseases. For example, researchers have found β-amyloid peptides and aggregates thereof in the supranucleus of the lens of the eyes of Alzheimer's disease [AD] victims (see U.S. Pat. No. 7,297,326 of Goldstein et al.) It has been shown that the presence of, or an increase in, the amount of β-amyloid peptides and aggregates thereof in the supranuclear and/or cortical lens regions of a test mammal's eye compared to a normal control value indicates that the test mammal is suffering from, or is at risk of developing, a neurodegenerative disease such as an amyloidogenic disorder (see WO 2012/024188). Since the supranucleus is only a fraction of a millimeter thick, measurements obtained from this region of the crystalline lens need to be accurate in location, specific in information and fast in acquisition. This is especially true because the human eye is in almost constant motion even when a patient is fixating on an illuminated target. Typically, eye movements must be compensated by techniques such as tracking or online image registration. Eye tracking methods are typically based on image analysis of features of the retina or the edge of the pupil. With a confocal optical system focused in the lens, concurrent imaging of the retina and/or pupil may not be possible.

There is an ongoing need for robust methods for permitting early detection of amyloidogenic disorders.

It is thus an object of the present invention to provide a method for measuring the amount of a fluorophore in ocular tissue.

The object underlying the present invention is solved by the subject matter of the present invention.

### SUMMARY OF THE INVENTION

This object is solved by the claimed subject matter, particularly by a method for measuring the amount of a fluorophore in ocular tissue, the method comprising the following steps:

a) contacting the ocular tissue with a first fluorophore that specifically binds to a protein;

b) illuminating the ocular tissue with a light source suitable to elicit fluorescence of the first fluorophore and suitable to elicit fluorescence of a second fluorophore, which is used as a reference;

c) determining a first light signal intensity for a selected lifetime (τ1) or a lifetime interval (dt1) of the fluorescence emitted by the first fluorophore and a second light signal intensity for a selected lifetime (τ2) or a lifetime interval (dt2) of the second fluorophore, wherein the first and second light signals are derived from the same region in the eye;

d) determining a ratio (r) of the first signal intensity to the second signal intensity, and

e) using the ratio (r) of the first to the second signal intensity for normalization of the determined light signal intensities.

Specifically, a method is provided by the invention, which allows correction for at least one of eye blinking or eye movement. According to the invention, the ratio (r) is used for normalizing fluorescence signals derived from a fluorophore in order to correct for measurement inaccuracies. By determining the ratio (r) as defined above, a highly accurate measurement of the actual amount of a fluorophore bound to a protein in ocular tissue can be achieved. In particular, the inventors have found that by using the method of the invention, the intensity of a fluorescent signal emitted by a fluorophore in ocular tissue can be measured independently of factors interfering with the measurement, such as eye blinking or eye movements. By determining the intensity of a first fluorescence signal derived from a first fluorophore (which typically directly correlates with the amount of protein, to which the fluorophore binds specifically) as well as the intensity of a second fluorescence signal derived from a second fluorophore, which is used as reference, the obtained fluorescence signal of the fluorophore can be corrected/normalized. It is thereby avoided that values are obtained as results, which are influenced by eye blinks and/or movements. The overall accuracy of the method is thus increased.

The method comprises illuminating the eye with a light source and measuring in the time domain the number of photons produced by natural fluorophores or exogenous fluorescent agents in the eye, and data normalization so as to correct for eye motion and blinking. The exogenous agent can be a molecule with binding characteristics to a certain protein indicative of a disease.

According to the method of the invention, the time-domain is used for discriminating between a first fluorescent signal emitted by a first fluorophore (such as an exogenous compound) on the one hand and a second fluorescent signal emitted by a second fluorophore (such as the ocular tissue, whose autofluorescence may be used as reference) on the other hand based on the difference in their respective fluorescence lifetimes. Fluorescence lifetime values are obtained by collecting photons in a time-dependent manner. Based on the arrival times of photons at a detector, light signals from a first fluorophore and from a second fluorophore having different fluorescence lifetimes may thus be differentiated.

The intensities of both fluorescent signals are measured in one single measurement in a single location over time. A histogram of photons is constructed as function of time. Based on the obtained histogram, curve fitting is performed with a multi-exponential decay curve. For each fluorophore, a lifetime value (τ) is retrieved from the curve.

Fluorescent intensities are obtained for a first fluorophore and a second fluorophore by measuring the fluorescent signals of a selected lifetime (τ1) value or a lifetime interval (dt1) and a selected lifetime value (τ2) or a lifetime interval (dt2). The lifetime intervals can be defined as set of lifetime values that include a peak value of the selected lifetime values. The lifetime interval further comprises the discrete time points corresponding to lifetime values, which fall within the full-width half maximum of the lifetime values. The interval boundaries are set so that there is no overlap between the two lifetime intervals. The lifetime intervals can be determined empirically. Furthermore, the lifetime intervals may also be derived from other experimental results (e.g. in vitro) and can also be further determined by an automated algorithm that searches for well defined peaks with a certain separation from each other.

A ratio (r) is then calculated of the value(s) obtained for the first fluorophore (e.g. an exogenous compound) to the value(s) obtained for the second fluorophore (e.g. a different exogenous compound or autofluorescence of ocular tissue as reference/background). Advantageously, the value of ratio (r) is not influenced by eye blinks or eye movements that take place during the measurement.

A distinct value for ratio (r) is preferably characteristic for a healthy subject, whereas another distinct value for ratio (r) is usually obtained in subjects, wherein the amount of the analyzed protein differs from the one observed in normal healthy individuals. The ratio (r) may be used—together with other clinical parameters—for aiding in diagnosis of a disease, which is associated with the presence of a protein, which is bound by the fluorophore that is administered to the ocular tissue. In some embodiments of the invention, the presence of such a protein or the amount of such protein in ocular tissue is indicative for a certain disease, e.g. an amyloidogenic disease. Typically, the ratio (r) is used as a threshold in order to distinguish amounts of said protein, which are usually found in healthy subjects, from protein amounts, which are usually found in subjects suffering from a disease. In contrast to the fluorescence signal emitted by a first fluorophore alone, the ratio (r) is invariant even if the subject blinks during measurement or if the subject's eye moves during the measurement.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic diagram of an optical device in accordance with an embodiment of the invention.

FIGS. 2A and 2B are graphs illustrating determination of fluorescence decay time in accordance with an embodiment of the invention.

FIG. 3 is a schematic diagram illustrating the use of time-correlation single photon counting, in accordance with an embodiment of the invention.

FIG. 4 is an example of a hypothetical array of fluorescence lifetimes used to normalize ophthalmological data, in accordance with an embodiment of the invention.

FIG. 5 shows the results (ratios-signal/background) obtained of two groups, in which a threshold ratio around 0.37 can discriminate between the groups, in a trial provided in an example.

### DETAILED DESCRIPTION OF THE INVENTION

In accordance with an embodiment of the invention, the method comprises illuminating the eye with a light source and measuring in the time domain the number of photons produced by natural fluorophores or exogenous fluorescent agents in the eye, and constructing a data normalization method to correct for eye motion or blinking. The exogenous fluorescent agent is preferably a molecule, which binds specifically to a protein in the eye. In some embodiments, the protein (or an increased amount of that protein) is indicative for a certain disease or condition.

In a preferred embodiment, the method comprises discriminating different fluorophores by their individual fluorescence lifetime and calculating the ratio (r) of their fluorescent signals, such as by taking one fluorescence signal as the signal and the other as the background/reference or as a normalization factor. Preferably, the ratio (r) is invariant within one subject, independently of an eye blink or a movement of the eye during measurement. In a preferred embodiment, the value of ratio (r) defines a threshold with respect to a critical amount of the protein, to which the first fluorophore binds specifically. Preferably, a characteristic value for (r) is obtained in subjects having normal (i.e. “healthy”) levels of the protein, whereas another, different value for ratio (r) is obtained in subjects, wherein that protein level is either increased or decreased (as in certain diseases).

The fluorescence data is collected in one single measurement in a single location within the eye, preferably in the lens, more preferably in the supranuclear and/or cortical region of the lense to obtain the fluorescence lifetime (τ1) and (τ2), respectively. In the case of aiding in the diagnosis of a disease by using an exogenous molecule, the ratio (r) is established of the first signal corresponding to the exogenous molecule to the second signal corresponding to a reference (e.g. autofluorescence of the ocular tissue).

In the context of the invention, the terms “fluorescence lifetime”, “lifetime”, “lifetime value”, “fluorescence decay time”, “fluorescence decay rate” and the like are used interchangeably. Generally, these terms are used as an indication of the time a fluorophore spends in the excited state before returning to the ground state by emitting a photon. Typically, the lifetimes of fluorophores range from picoseconds to hundreds of nanoseconds. More specifically, the term “fluorescence lifetime” as used herein relates to the parameter τ, which indicates the time it takes for the number of excited molecules to decay to 1/e or approximately 36.8% of the original population. τ differs between the first and the second fluorophor as used in the method of the invention. Preferably, τ differs also between a compound, which is unbound, and the same compound, which is bound to, e.g., a protein, making it possible to distinguish bound and unbound fluorophore on the basis of the fluorescence decay rate.

The lifetime intervals (dt1) or (dt2), over which the first fluorescence signal and the second fluorescence signal, respectively, are determined, comprise discrete time points corresponding to lifetime values, which fall within the full-width half maximum of lifetime values. According to the invention, the first and second light signals are determined for life time intervals, which are selected to comprise the respective lifetime value corresponding to the maximum total number of photons in an array. The light signal may be determined by using the peak lifetime value (or the respective photon counts) within the lifetime interval. Alternatively, the photon counts corresponding to the sum of discreste lifetime values in a lifetime interval dt1 or dt2 can be employed for determining the signal. An average or median lifetime value may further be calculated based on the discrete lifetime values within the lifetime interval in order to determine a light signal. In the meaning of the present invention, the peak lifetime values or calculated values as described above may equally be used for determining the light signals, on the basis of which the ratio is calculated.

In a preferred embodiment, a first lifetime interval (dt1) comprises lifetime values in the range from 2 to 2.8 nsec, preferably from 2.2 to 2.6 nsec, more preferably in the range from 2.3 to 2.5 nsec. In a preferred embodiment, a first lifetime value (τ1) is 2.4 nsec. In a further preferred embodiment, a second lifetime interval (dt2) comprises lifetime values in the range from 3.6 to 4.4 nsec, preferably from 3.8 to 4.2 nsec, more preferably in the range from 3.9 to 4.1 nsec. In a preferred embodiment, a second lifetime value (τ2) is 4.0 nsec.

The eye is contacted with the first fluorophore, which is administered to the eye at least 2 hours, preferably at least 4 hours, more preferably at least 8 hours, even more preferably at least 12 hours and most preferably at least 18 hours prior to the measurement of fluorescence. Administration may be direct (e.g. by way of an ophthalmic ointment) or indirect (e.g. by systemic administration) by using any suitable formulation. In one embodiment, the second fluorophore, which is used as a reference, is an endogenous fluorophore, such as an endogenous molecule comprised in the ocular tissue. In an alternative embodiment, the second fluorophore is an exogenous fluorophore (distinct from the first fluorophore), which is administered to the eye before, after or during the contacting of the eye with the first fluorophore, in such a manner that both fluorophores are concomittantly present in the eye.

In accordance with an embodiment of the invention, there is provided a method for improving the molecular contrast in fluorescence measurements in ocular tissue of subjects suffering from a disease, which may be an ocular disease, such as age-related macular degeneration; an amyloidogenic disorder, such as Alzheimer's Disease; or a pre-morbid neurodegenerative state. The disease can involve the development of beta amyloid aggregates in the eye, and in particular, in the supranuclear region of the lens in the eye. The method is carried out by illuminating an ocular tissue in a mammal, e.g., a human subject, preferably with a pulsed laser source.

The method may further comprise comparing the ratio to a predetermined threshold ratio indicative of a disease condition for aiding in diagnosis of said disease or condition; and/or assigning a probability of a disease condition based on the ratio together with other clinical parameters; and/or assigning a value corresponding to extent of progression of a disease condition based on the ratio together with other clinical parameters; and/or assigning a value corresponding to extent of progress of treatment of a disease condition based on the ratio as well as other clinical parameters. Typically, determining the ratio (r) is by itself not sufficient for diagnosis but is taken together with other clinical signs. At least one of the first fluorescence lifetime and the second fluorescence lifetime may comprise a fluorescence lifetime of a signal indicative of a disease condition manifested at least in part in the ocular tissue, and the disease condition may comprise at least one of: an ocular disease; an amyloidogenic disorder and a pre-morbid neurodegenerative state. In a preferred embodiment, the disease is selected from the group consisting of Alzheimer's disease (AD), familial AD, Sporadic AD, Creutzfeld-Jakob disease, variant Creutzfeld-Jakob disease, spongiform encephalopathies, Prion diseases (including scrapie, bovine spongiform encephalopathy, and other veterinary prionopathies), Parkinson's disease, Huntington's disease (and trinucleotide repeat diseases), amyotrophic lateral sclerosis, Down's Syndrome (Trisomy 21), Pick's Disease (Frontotemporal Dementia), Lewy Body Disease, neurodegeneration with brain iron accumulation (Hallervorden-Spatz Disease), synucleinopathies (including Parkinson's disease, multiple system atrophy, dementia with Lewy Bodies, and others), neuronal intranuclear inclusion disease, tauopathies (including progressive supranuclear palsy, Pick's disease, corticobasal degeneration, hereditary frontotemporal dementia (with or without Parkinsonism), a pre-morbid neurodegenerative state and Guam amyotrophic lateral sclerosis/parkinsonism dementia complex). Preferably, the disease condition is Alzheimer's Disease.

In further related embodiments, the method may comprise determining the ratio at each of a plurality of time points for a single subject's eye, and determining an average ratio for the single subject based on the ratio at the plurality of time points. The method may comprise determining at least one of the first light signal intensity and the second light signal intensity based on at least one of a pixel weighted photon count over the area of the ocular tissue and an average photon count over the area of the ocular tissue. The first light signal intensity may comprise a first peak value of fluorescence intensity of the first photons assigned to the first fluorescence lifetime, and the second light signal intensity may comprise a second peak value of fluorescence intensity of the second photons assigned to the second fluorescence lifetime. The first light signal intensity may comprise a first value corresponding to the number or frequency of photons having a fluorescence lifetime (τ1) within a first lifetime interval (dt1), and the second light signal intensity may comprise a second value corresponding to the number or frequency of photons having a fluorescence lifetime (τ2) within a second lifetime interval (dt2).

In further related embodiments, the method may comprise illuminating the ocular tissue with a light source, thereby inducing emission of a plurality of photons. The light source may have at least one of a wavelength property, a polarization property or a combination thereof, each appropriate to produce fluorescence in the ocular tissue; and the method may further comprise receiving light including fluorescence produced as a result of the illuminating the eye, and determining the first fluorescence lifetime for the first fluorophore and the second fluorescence lifetime for the second fluorophore based on the received light, preferably based on the arrival time of the emitted light at a photo detector. The method may further comprise performing a time correlation single photon count based on received electrical signals indicative of photon counts of the fluorescence produced as a result of illuminating the eye. The light source may comprise a pulsed light source, such as a femto-second to nano-second pulsed light source. The method may comprise illuminating the ocular tissue with multiple wavelengths of light in a single measurement. Preferably, the light source is a pulsed laser beam.

In a further preferred embodiment, the light source may be configured to emit light of an appropriate wavelength for a peak region of a fluorescent excitation spectrum for a fluorophore in the eye, and an optical scanning system may be configured to detect light of an appropriate wavelength for a peak region of a fluorescent emission spectrum for the fluorophore and/or the autofluorescence of the ocular tissue. For example, the excitation spectrum may have a peak between 400 nm and 500 nm, preferably of about 470 nm, the light source being configured to emit light within plus or minus about 20 nm of the peak of the excitation spectrum, and the emission spectrum may have, for instance, a peak between 500 nm and 600 nm, preferably at about 580 nm, the optical scanning system being configured to detect light within plus or minus about 20 nm of the peak of the emission spectrum. The repetition rate of the pulsed laser is preferably from 30 to 70 MHz, more preferably from 40 to 60 MHz, most preferably from 45 to 55 MHz. In a preferred embodiment, the repetition rate of the laser pulse is 50 MHz.

In an alternative embodiment, a second light source can be used, e.g. in cases where multiple fluorophores have different absorption spectra. For instance, one laser can be used for exciting a first fluorophore and a second laser to excite a second fluorophore. The lifetimes of the the first (τ1) and the second fluorophores (τ2) can then be determined.

A suitable optical scanning system preferably enables the detection of fluorescent molecules and differentiation between them based on their optical signatures, such as fluorescence decay time (τ). In a preferred embodiment, the method according to the invention is carried out by using a fluorescence scanning mechanism combined with fluorescence lifetime spectroscopy in order to enable the detection of fluorescent molecules and to provide information on their spatial distribution. The system may determine a location of an ocular interface (such as a lens capsule) of the eye based on an increase in natural fluorescence emitted from tissues. A scan with a set of galvanometer mirrors is performed within the lens and photons are collected in time. The scan is divided into an array of pixels where collected photons are binned according to their arrival time, i.e. sections of the scan area are combined depending on their arrival time at the detector. A lifetime histogram of photon arrivals is constructed for each pixel and lifetime values are assigned.

In an embodiment according to the invention, fluorescence excitation is achieved by a pulsed laser beam and is focused by a high numerical aperture objective lens into the eye. The arrival of photons at the detector (for example, an avalanche photodiode detector) is time stamped using a time correlation single photon counting data acquisition board. Lifetime values are extracted over the scanned area. The light signal intensity corresponding to the fluorescence lifetime value of a fluorophore or to a lifetime interval (e.g., 2.4 nsec±0.4) is assigned as “signal.” The light signal intensity corresponding to the lifetime value of autofluorescence (e.g., 4 nsec±0.4) is designated as “background” or “reference”.

In a preferred embodiment of the invention, the lifetime value (τ1) of a first fluorophore, which is emitting a first light signal, and the lifetime value (τ2) of a second fluorophore (e.g. autofluorescence of the ocular tissue as “background”), which is emitting a second light signal, differ by at least 0.3 nsec, preferably by at least 0.4 nsec, more preferably by at least 0.5 nsec, even more preferably by at least 1 nsec and most preferably by at least 1.5 nsec.

According to the invention, ocular tissue is contacted with a fluorophore, which binds specifically to a protein. Preferably, the first fluorophore binds specifically to a protein; whose presence in ocular tissue is indicative for a certain disease. More preferably, the first fluorophore binds to a protein, which is indicative for a certain disease if its amount is above or below a threshold that has been pre-defined for a certain disease.

In a preferred embodiment, the first fluorophore binds to a protein, whose presence in the eye is indicative for an amyloidogenic disease. Preferably, fluorophores, which are bound to an amyloid protein in the eye, can be distinguished from unbound fluorophores due to their distinct fluorescence decay rate.

In another preferred embodiment, the first fluorophore binds to an amyloid protein, such as β-amyloid (Aβ). By “amyloid protein,” it is meant a protein or peptide that is associated with an AD neuritic senile plaque, regardless of whether the amyloid protein is aggregated (fully or partially). Preferably, the amyloid protein is amyloid precursor protein (APP) or an (e.g., naturally-occurring) proteolytic cleavage product of APP such as Aβ. APP cleavage products include Aβ1-40, Aβ2-40, Aβ1-42, as well as oxidized or crosslinked Aβ. The fluorophore may also bind to naturally-occurring variants of APP and AP, including single nucleotide polymorphic (SNP) variants. The fluorophore may, but need not necessarily, bind to β-amyloid aggregate. A discussion of fluorophore binding to β-amyloid aggregates may be found in Goldstein et al., “Cytosolic β-amyloid deposition and supranuclear cataracts in lenses from people with Alzheimer's disease,” Lancet 2003; 361: 1258-65.

For example, the method according to the invention can utilize amyloid-binding fluorescent molecular rotor compounds to detect amyloid peptides in the eye. Examples of fluorescent molecular rotor compounds that have been used to analyze brain tissue (but not eye tissue) include X-34 and {(trans, trans)-1-bromo-2,5-bis-(3-hydroxycarbonyl-4-hyrdoxy)styrlbenzene (BSB)} (Styren et al., 2000, J. Histochem., 48:1223-1232; Link et al., 2001, Neurobiol. Aging, 22:217-226; and Skovronsky et al., 2000, Proc. Natl., Acad. Sci. U.S.A., 97(13):7609-7614). These fluorescent molecular rotor compounds emit light in the blue-green range, thus the level of fluorescence, which is diagnostically relevant, exceeds the amount of human lens autofluorescence in the blue-green range. For example, other useful fluorescent molecular rotor compounds include Me-X04 (1,4-bis(4′-hydroxystyryl)-2-methoxybenzene), Chrysamine or Chrysamine derivative compounds such as {(trans, trans)-1-bromo-2,5-bis-(3-hydroxycarbonyl-4-hyrdoxy)styrlbenzene (BSB)}. Such compounds are described in Mathis et al., Curr. Pharm. Des., 10(13):1469-93 (2004); U.S. Pat. Nos. 6,417,178; 6,168,776; 6,133,259; and 6,114,175. Nonspecific amyloidphilic fluorescent molecular rotor compounds such as thioflavin T, thioflavin S or Congo red dye may also be used. For example, the following structural formulas may be suitable fluorescent molecular rotor compounds:

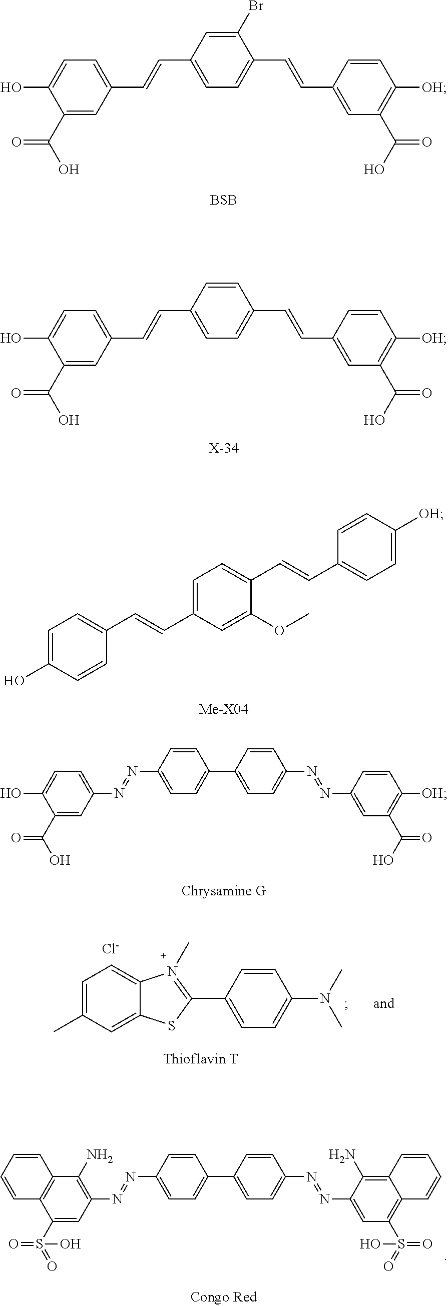

In the context of the present invention, the term “compound” also comprises pharmaceutically acceptable salts of the compounds as defined herein. The phrase “pharmaceutically acceptable salt(s)”, as used herein, refers to salts of compounds of the invention that are safe and effective for use in mammals and that possess the desired biological activity. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. In a preferred embodiment, a compound of the invention can form a pharmaceutically acceptable salt with an amino acid. Suitable base salts include, but are not limited to, aluminum, calcium, lithium, magnesium, potassium, sodium, zinc, and diethanolamine salts. Preferably, a pharmaceutically acceptable salt of a compound according to the invention is a hydrohalogenide salt, more preferably a hydrochloride or hydrobromide salt and most preferably a hydrochloride salt.

In one embodiment, a fluorescent molecular rotor compound is used as a fluorophor, which is represented by structural Formula (I), or a pharmaceutically acceptable salt thereof:

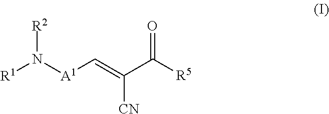

wherein:

A¹ is an optionally substituted C6-C18 arylene, an optionally substituted C5-C18 heteroarylene, or is represented by the following structural formula:

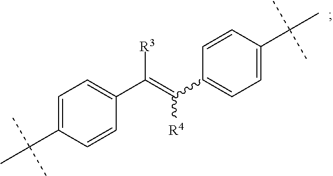

R¹ and R² are each independently hydrogen, optionally substituted C1-C12 alkyl, an optionally substituted C1-C12 heteroalkyl, optionally substituted C3-C12 cycloalkyl, or R¹ and R² taken together with the nitrogen atom to which they are attached form an optionally substituted 3 to 12 membered heterocycloalkyl;

R³ and R⁴ are each independently hydrogen, methyl, or ethyl;
R⁵ is —OH, optionally substituted —O(C1-C6 alkyl), —NR⁶R⁷, or is represented by the following structural formula:

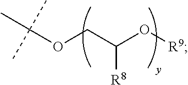

R⁶ and Ware each independently, hydrogen, methyl, ethyl, or R⁶ and R⁷ taken together with the nitrogen atom to which they are attached form a 5 to 7 membered heterocycloalkyl containing one to three ring heteroatoms independently selected from N, O, and S;

wherein:

y is an integer from 1 to 10;

R⁸, for each occurrence independently, is hydrogen, —OH, or —CH₂OH;

R⁹ is hydrogen, —NR¹⁰R¹¹, —C(O)R¹², optionally substituted C1-C6 alkyl, or optionally substituted C1-C6 heteroalkyl;

R¹⁰, R¹¹ and R¹² are each independently hydrogen or C1-C6 alkyl.

In some embodiments, A¹ is selected from the group consisting of an optionally substituted phenyl, an optionally substituted naphthyl, an optionally substituted (E)-stilbene, or an optionally substituted (Z)-stilbene. In another embodiment, A¹ is an optionally substituted naphthyl. Values and preferred values of the remainder of the variables are as defined above and below with respect to Formula (I).

In a preferred embodiment, a fluorescent molecular rotor compound is used as a fluorophore, which has the structural Formula (II). The compound of Formula (II) is a compound of Formula (I), wherein A¹ is represented by the following structural formula:

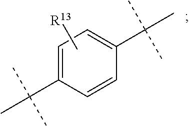

and is represented by the following structural Formula (II), or a pharmaceutically acceptable salt thereof:

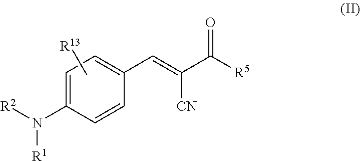

wherein:

R¹³ is hydrogen, —OH, or optionally substituted —O(C1-C6 alkyl).

Values and preferred values of the remainder of the variables are as defined above and below with respect to Formula (I).

In a preferred embodiment, the fluorescent molecular rotor compound, which is used as a fluorophor, is a compound according to structural Formula (III). The compound of Formula (III) is a compound of Formula (I), wherein A¹ is represented by the following structural formula:

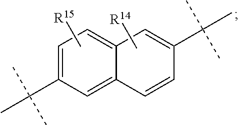

and is represented by the following structural Formula (III), or a pharmaceutically acceptable salt thereof:

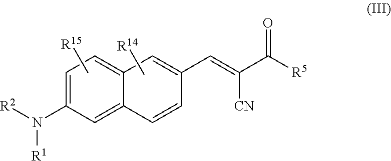

wherein:

R¹⁴ and R¹⁵ are each independently hydrogen, —OH, or optionally substituted —O(C1-C6 alkyl).

Values and preferred values of the remainder of the variables are as defined above and below with respect to Formula (I).

In some embodiments, R¹ and R² are both optionally substituted C1-C12 alkyl. In other embodiments, R¹ and R² are both selected from the group consisting of methyl, ethyl, propyl, and butyl. Values and preferred values of the remainder of the variables are as defined above and below with respect to Formula (I).

In some embodiments, R¹ and R² taken together with the nitrogen atom to which they are attached form an optionally substituted 3 to 12 membered heterocycloalkyl. In another embodiment, R¹ and R² taken together with the nitrogen atom to which they are attached form heterocycloalkyl selected from the group consisting of piperidine, morpholine, piperazine, and 1-methylpiperazine. Values and preferred values of the remainder of the variables are as defined above and below with respect to Formula (I), Formula (II), or Formula (III).

In some embodiments, R⁵ is

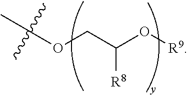

Values and preferred values of the remainder of the variables are as defined above and below with respect to Formula (I), Formula (II), or Formula (III).

In some embodiments, R⁵ is

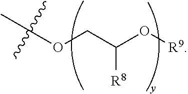

y is 1;

R⁸ is —CH₂OH; and

R⁹ is —OH.

Values and preferred values of the remainder of the variables are as defined above and below with respect to Formula (I), Formula (II), or Formula (III).

In some embodiments, R⁵ is

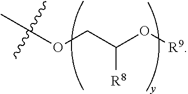

y is 3; and

R⁹ is methyl.

Values and preferred values of the remainder of the variables are as defined above and below with respect to Formula (I), Formula (II), or Formula (III).

In some embodiments, R⁵ is

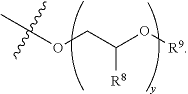

y is 4; and

R⁹ is methyl.

Values and preferred values of the remainder of the variables are as defined above and below with respect to Formula (I), Formula (II), or Formula (III).

In some embodiments, A¹ is selected from the group consisting of an optionally substituted phenyl, an optionally substituted naphthyl, an optionally substituted (E)-stilbene, or an optionally substituted (Z)-stilbene; R¹ and R² are both optionally substituted C1-C12 alkyl; and R⁵ is

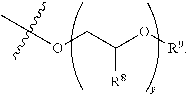

Values and preferred values of the remainder of the variables are as defined above and below with respect to Formula (I).

In some embodiments, A¹ is selected from the group consisting of an optionally substituted phenyl, an optionally substituted naphthyl, an optionally substituted (E)-stilbene, or an optionally substituted (Z)-stilbene; R¹ and R² taken together with the nitrogen atom to which they are attached form an optionally substituted 3 to 12 membered heterocycloalkyl; and R⁵ is

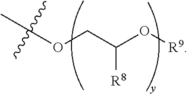

Values and preferred values of the remainder of the variables are as defined above and below with respect to Formula (I).

In some embodiments, A¹ is an optionally substituted phenyl; R¹ and R² are both optionally substituted C1-C12 alkyl; and R⁵ is

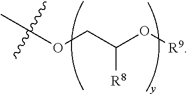

Values and preferred values of the remainder of the variables are as defined above and below with respect to Formula (I).

In some embodiments, A¹ is an optionally substituted phenyl; R¹ and R² taken together with the nitrogen atom to which they are attached form an optionally substituted 3 to 12 membered heterocycloalkyl; and R⁵ is

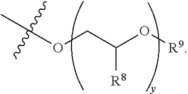

Values and preferred values of the remainder of the variables are as defined above and below with respect to Formula (I).

In some embodiments, A¹ is an optionally substituted naphthyl; R¹ and R² are both optionally substituted C1-C12 alkyl; and R⁵ is

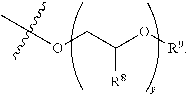

Values and preferred values of the remainder of the variables are as defined above and below with respect to Formula (I).

In some embodiments, A¹ is an optionally substituted naphthyl; R¹ and R² taken together with the nitrogen atom to which they are attached form an optionally substituted 3 to 12 membered heterocycloalkyl; and R⁵ is

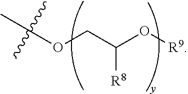

Values and preferred values of the remainder of the variables are as defined above and below with respect to Formula (I).

In some embodiments, the fluorescent molecular rotor compound is selected from the group consisting of:

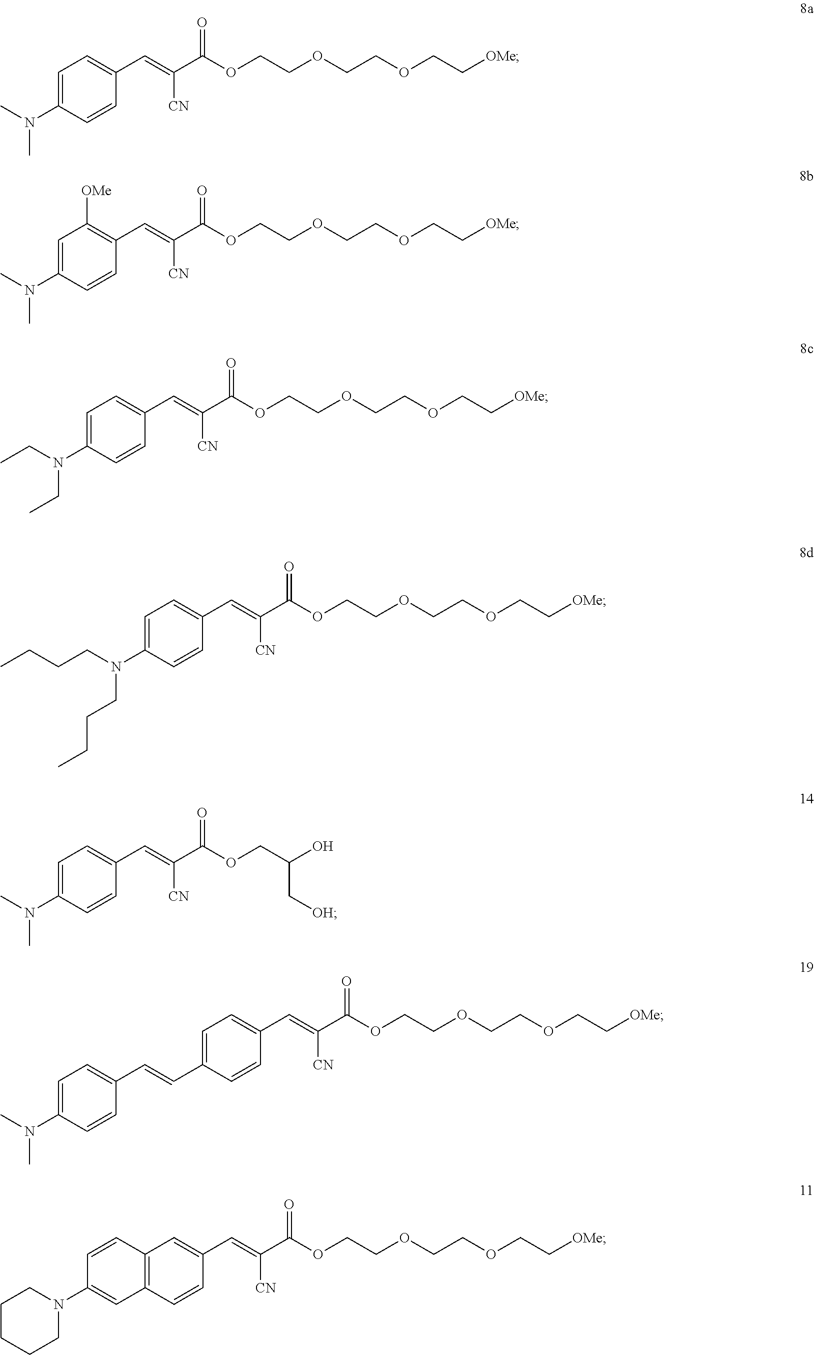

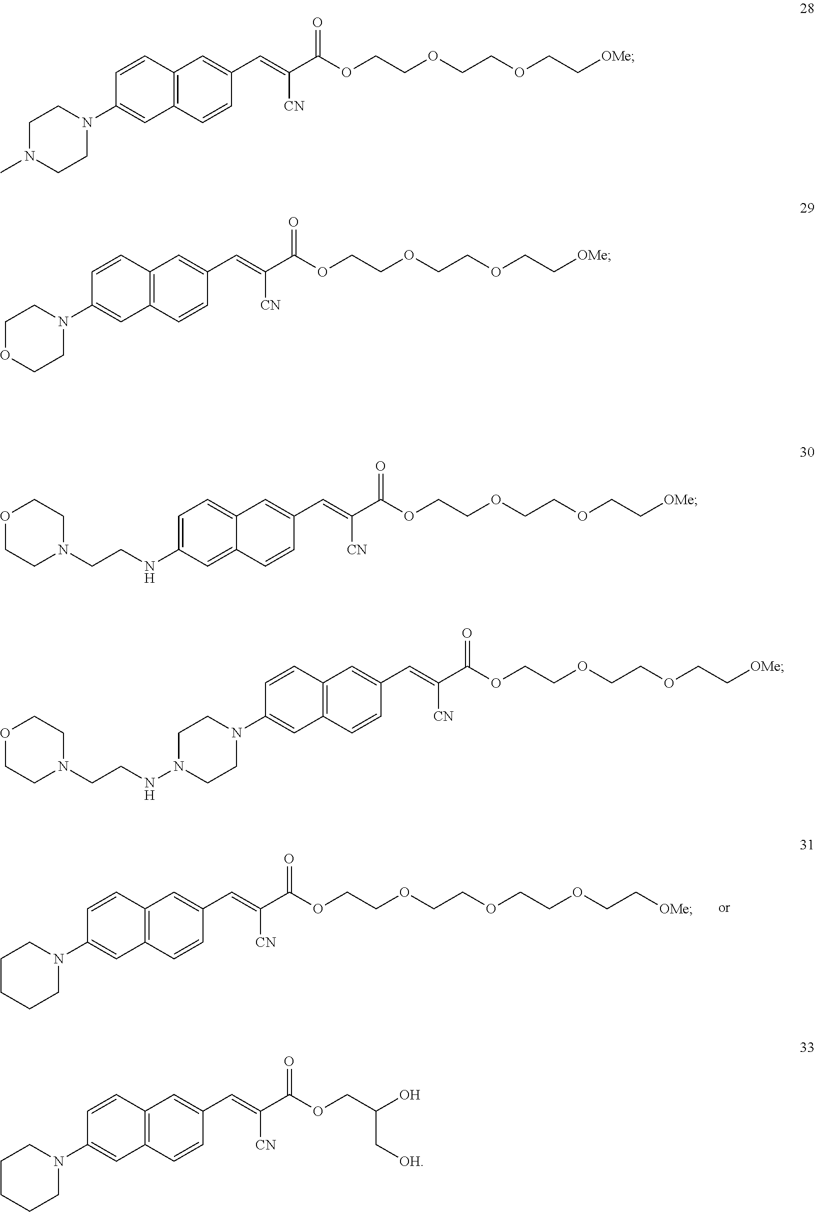

In some embodiments, the method according to the invention uses as a fluorophor a compound of the following structural Formula (I), structural Formula (II), or structural Formula (III), or a pharmaceutically acceptable salt thereof:

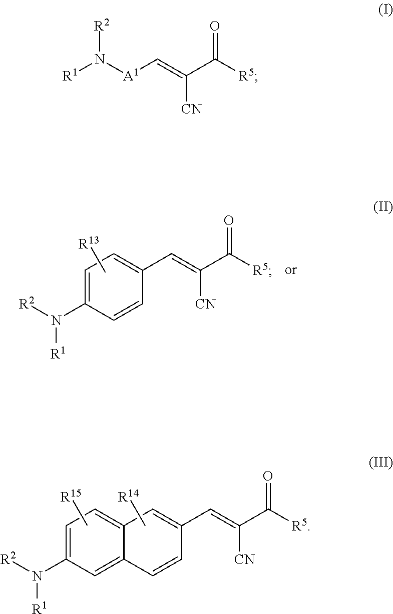

The fluorescent molecular rotor compounds of structural Formula (I) can be synthesized by any methods known to those of skill in the art. For example, suitable fluorescent molecular rotor compounds can be synthesized by the methods described in PCT Publication WO 2011/072257.

In a particularly preferred embodiment, the method according to the invention comprises the use of a compound having the structural formula

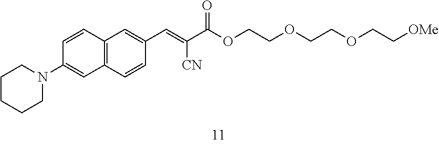 [SMILES: COCCOCCOCCOC(=O)/C(C#N)=C/c1ccc2cc(N3CCCCC3)ccc2c1]

or a pharmaceutically acceptable salt as defined herein as a first fluorophor, which binds to an amyloid protein in the ocular tissue. In the context of the present invention, the above compound is also referred to as compound #11 or aftobetin. Preferably, aftobetin or its hydrohalogenide salt is used in the method of the invention. In a further preferred embodiment, the hydrochloride salt of compound #11 (also referred to as “compound #11-HCl”, “aftobetin hydrochloride” or “aftobetin-HCl”) is used. In a further preferred embodiment, the method comprises the use of the above compound #11 (aftobetin) or a pharmaceutically acceptable salt thereof as a first fluorophore and the autofluorescence of the ocular tissue as second fluorophore/reference. Compound #11 or a pharmaceutically acceptable salt thereof may be administered to the eye (e.g. by way of an ophthalmic ointment or other suitable administration routes) before the measurement. In a preferred embodiment, compound #11 or a pharmaceutically acceptable salt thereof is administered to the eye at least 2 hours, preferably at least 4 hours, more preferably at least 8 hours, even more preferably at least 12 hours and most preferably at least 18 hours prior to the measurement of fluorescence. In a preferred embodiment, compound #11 or a pharmaceutically acceptable salt thereof is administered to the eye at least 18 hours prior to fluorescence measurements, wherein virtually no unbound compound #11 is present in ocular tissue at the time of fluorescence measurement. The amount of compound #11 or a pharmaceutically acceptable salt thereof bound to amyloid protein in ocular tissue is determined by fluorescence measurement, preferably in the supranuclear and/or cortical region of the lens. The fluorescence decay rate of compound #11 or a pharmaceutically acceptable salt thereof (τ1, e.g. 2.4 nsec+/−0.4 nsec) is distinct from the decay rate of the autofluorescence of the ocular tissue (τ2, e.g. 4 nsec+/−0.4 nsec), making it possible to distinguish the specific signal from the autofluorescence of the ocular tissue (background), which is used as a reference. By performing the ratio (r) between the values obtained for compound #11 or a pharmaceutically acceptable salt thereof and the reference, respectively, a normalization is performed in order to correct for eye blinks or movements.

In further related embodiments, the method may comprise constructing a histogram of photon counts received as a function of time; fitting a multi-exponential decay curve to the histogram; and retrieving at least the first fluorescence lifetime and the second fluorescence lifetime from time decay rates of first and second respective component exponential decay curves of the multi-exponential decay curve.

In accordance with an embodiment of the invention, a method may include the following steps:

1) A histogram of photons detected is constructed as function of time.

2) A fitting curve of the histogram is performed with a multi-exponential decay curve.

3) Lifetime values τ1 and τ2 are retrieved from the curve.

4) For each lifetime, a value (for example, number of photons) is assigned in an array of elements where each value within the element is sorted to the n-th bin of the array.

5) The value (for example, number of photons) in each element is weighted to number of photons.

6) A summation of all values of interest is made (such as each of the signal and the background).

7) Measurement of a peak value within the range of the signal (e.g., 2.4 nsec±0.4).

8) Measurement of a peak value within the range of the background (e.g., 4 nsec±0.4).

9) Performing a ratio (r) of signal to background.

The ratio of the signal to background is used as a value to be compared with a predetermined threshold value of the ratio, which—together with other clinical parameters—permits discrimination between disease groups. For example, when the ratio exceeds the predetermined threshold value, a subject whose eye was measured may be assigned to an “Alzheimer's Disease” group on the basis of this result in combination with further clinical parameters indicative of Alzheimer's disease. On the other hand, when the ratio does not exceed the predetermined threshold value, the subject may be assigned to a “healthy” group in absence of other clinical signs.

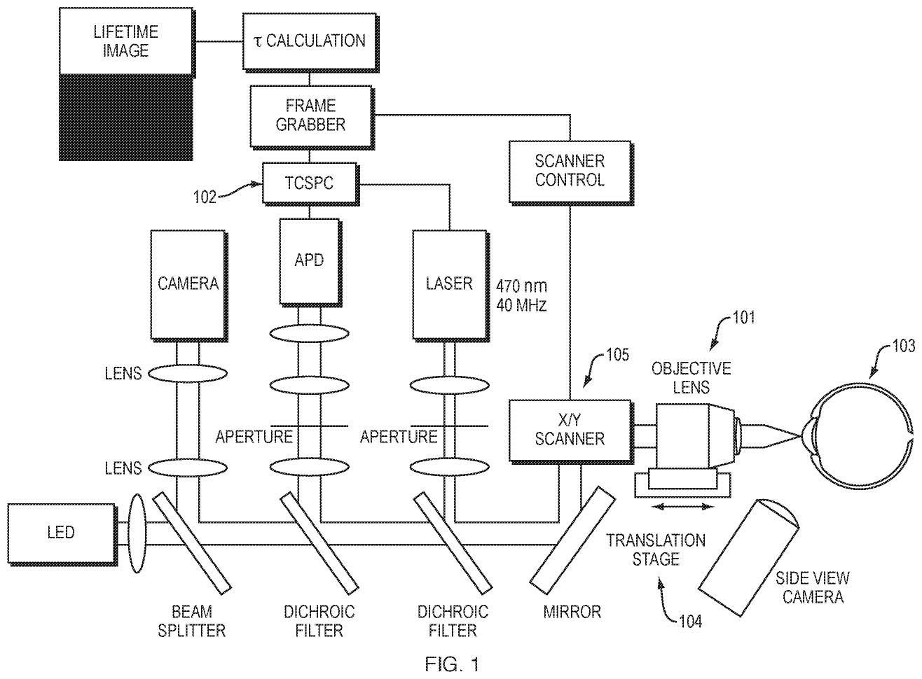

FIG. 1 is a schematic diagram of an optical device in accordance with an embodiment of the invention. Fluorescence excitation is achieved by a pulsed laser beam that is focused by a high numerical aperture objective lens 101 into the eye. Fluorescence is detected using a time correlation single photon counting (TCSPC) technique through a confocal configuration with a fast avalanche photodiode detector (APD) 102. TCSPC is performed by using a short pulse of light to excite the sample (eye) 103 repetitively, and recording the subsequent fluorescence emission as a function of time. This usually occurs on the nanosecond timescale.

In the embodiment of FIG. 1, identification of the anatomical structures of the lens is performed by scanning the objective lens 101 on axis using a translation stage 104. The signal is measured at every point along the scan in order to reveal the anatomical structures of the anterior segments such as the cornea, lens capsule and supranucleus region of the lens. In addition, the scan provides information about the pharmaco-kinetics of exogenous amyloid-binding compounds applied to the eye. Such information provides not only spatial and temporal information of the amyloid-binding compound, but also the concentration of the amyloid-binding compound that penetrates through the cornea and into the aqueous humor.

In the embodiment of FIG. 1, once the location of interest in the eye is known from the excited natural fluorescence measured at every point along the axial scan, another scan is executed in a plane (xy) perpendicular to the optical axis using a set of galvanometer mirrors 105. To ensure allocation of the measured fluorescence decay curves to the corresponding site of the two-dimensional scanning, the galvanometer set scanning is synchronized with the laser pulses and photodetection for time-correlated individual photon counting. In the embodiment of FIG. 1, one or more modules may be implemented using dedicated, specialized hardware modules and/or using a general purpose computer specially-programmed to perform the modules' functionality, including, for example, the Frame Grabber module, TCSPC module, τ Calculation module and scanner control module. A general purpose computer and/or one or more specialized hardware modules may receive data from each other via data cables and data ports appropriate for the modules' functionality.

In the embodiment of FIG. 1, for time-correlated individual photon counting, the decay curve of the autofluorescence is registered for each scanned location of the lens and thus a two-dimensional representation of the fluorophores' distributions can be evaluated and analyzed based on their fluorescence decay time as well as on their intensity. The image of the calculated decay times can be encoded by false colors and can be superimposed on the intensity image for better clinical interpretation. Since the fluorescence decay time is a characteristic for each fluorescence molecule, one can determine and separate the fluorophores (amyloid-binding compound from natural fluorescence of the lens) being excited in the sample volume. By combining fluorescence intensity and lifetime measurements, an extra dimension of information is obtained to discriminate among several fluorescent labels.

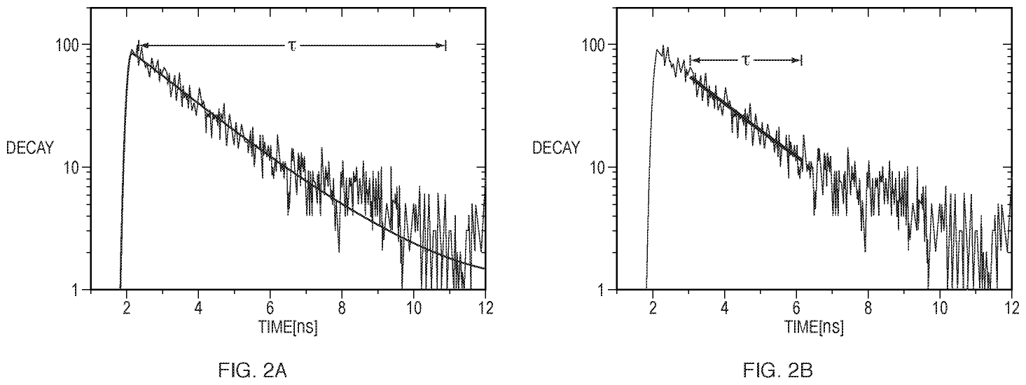

FIGS. 2A and 2B are graphs illustrating determination of fluorescence decay time in accordance with an embodiment of the invention. Fluorescence decay lifetime may be calculated by a single or double fit exponential (FIG. 2A) to a curve of intensity (here, in photons/sec), versus time (here, in nanoseconds). It can be also obtained by a linear fit to the slope (FIG. 2B). As used herein, a “time decay rate of fluorescence” signifies a characteristic time constant of a decay curve of fluorescence intensity; for example, an exponential time constant or a slope fitted to the fluorescence decay curve.

The above algorithms of FIGS. 2A, 2B may, for example, be implemented using dedicated, specialized hardware modules and/or using a general purpose computer specially-programmed to perform the above algorithms. Such modules may, for example, use or receive data from the TCSPC module, Frame Grabber module, τ-calculation module of the embodiment of FIG. 1.

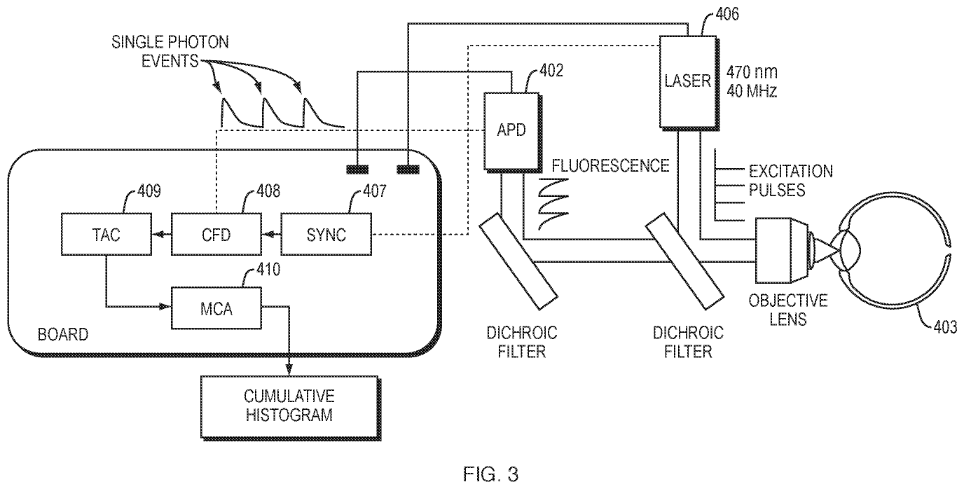

FIG. 3 is a schematic diagram illustrating the use of time-correlation single photon counting, in accordance with an embodiment of the invention. A pulsed light source 406 excites the sample 403 repetitively. The sample emission is observed by a detector unit avalanche photodiode (APD) 402, while the excitation flashes are detected by a synchronization module (SYNC) 407. A constant fraction discriminator (CFD) 408 responds to only the first photon detected—independent of its amplitude—from the detector 402. This first photon from sample emission is the stop signal for the Time-to-Amplitude Converter (TAC) 409. The excitation pulses trigger the start signals. The Multi-Channel Analyzer (MCA) 410 records repetitive start-stop signals of the single-photon events from the TAC 409, to generate a histogram of photon counts as a function of time channel units. The lifetime is calculated from this histogram. The MCA may be implemented using a dedicated, specialized hardware module and/or using a general purpose computer specially-programmed to perform such tasks; and may be in data communication with a specially-programmed general purpose computer.

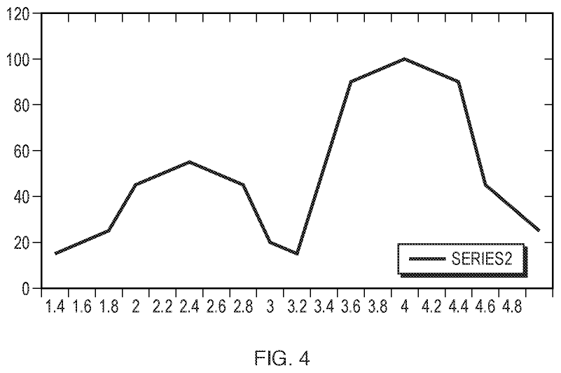

FIG. 4 is an example of a hypothetical array of fluorescence lifetimes used to normalize ophthalmological data, in accordance with an embodiment of the invention. Photon Count units, on an arbitrary scale (which may correspond to a scaled multiple of total photon counts), are shown on the y-axis, while fluorescence lifetimes are shown on the x-axis. In the example of FIG. 4, it can be seen that peaks are found at 2.4 nsec and 4.0 nsec. In an embodiment according to the invention, such peaks may be used to normalize the data. A peak value can be determined as a maximum of photon counts assigned to a lifetime value, where the signal is the largest value within a lifetime interval and does not overlap with the lifetime interval of the second signal.

In particular, the photon count units may be used as a measure of the fluorescence intensity for two fluorescence lifetimes: one, at the left hand peak of 2.4 nsec, corresponds to a “signal,” for example, fluorescence from a fluorescent ligand bound to amyloid beta protein; and the second, in the right hand peak at 4.0 nsec, corresponds to background autofluorescence of the eye. In an embodiment according to the invention, the measure of fluorescence intensity, such as the photon count unit measurements at the peaks, are used to determine a ratio. Here, for example, a ratio of 55 photon count units divided by 100 photon count units, or about 0.55, is found for the left hand peak's photon count of 55 divided by the right hand peak's photon count of 100. Thus, the ratio of the fluorescence intensity for the signal (here, 55 for the left peak at a lifetime of 2.4 nsec) over the fluorescence intensity for the background (here, 100 for the right peak at a lifetime of 4.0 nsec) is taken. Other techniques may be used than using only the exact peak: for example, all photon counts within a certain range (dt1) and (dt2) such as plus or minus 0.4 nsec, of each peak, may be summed for the purpose of forming a value for a first peak (the signal), which is then compared to a corresponding value for the other peak (the background) to determine a ratio. An average, a weighted average over pixels, or other measures of the fluorescence intensity may be used. Once the ratio is obtained, it may then be compared statistically against predetermined known statistics for the ratio for disease groups. For example, a diagnosis of Alzheimer's disease may be found for a ratio of greater than a predetermined ratio. Alternatively, a probability of a disease condition may be determined, or an estimated progress of a disease, or an estimate of progress of treatment of the disease, based on the ratio. Other techniques set forth in the summary, description and items herein may be used.

It will be appreciated that as used herein, the term “first photons” and “second photons” should not be taken as referring to the order of arrival of the photons, but rather purely in the categorical sense of labeling the two groups of photons as belonging to one of two groups (the “first” group and the “second” group), for example two groups with different characteristic fluorescence lifetimes.

The invention is further illustrated by the embodiments specified in the following items:
1. A method for imaging ocular tissue, the method comprising:
   determining a first measure of fluorescence intensity of first photons assigned to a first fluorescence lifetime, the first photons having been emitted from an area of the ocular tissue;
   determining a second measure of fluorescence intensity of second photons assigned to a second fluorescence lifetime, the second photons having been emitted from the same area of the ocular tissue; and
   determining a ratio of the first measure to the second measure.
2. The method of item 1, wherein determining the first measure and the second measure comprises constructing a distribution histogram of photons of a plurality of fluorescence lifetimes, and determining the first measure and the second measure based on the distribution histogram.
3. The method according to item 1 or 2, the method comprising thereby correcting for at least one of eye blinks and eye motion in the imaging of the ocular tissue.
4. The method according to any preceding item, wherein the first fluorescence lifetime comprises a fluorescence lifetime of a signal indicative of a disease condition manifested at least in part in the ocular tissue, and wherein the second fluorescence lifetime comprises a fluorescence lifetime of autofluorescence of the ocular tissue.
5. The method according to item 4, wherein the fluorescence lifetime of the signal indicative of the disease condition comprises a fluorescence lifetime of at least one of:
   an amyloid-binding compound;
   the amyloid-binding compound bound to an amyloid protein; and
   the amyloid protein.
6. The method of any preceding item, wherein the first measure of fluorescence intensity comprises a first photon count of the first photons assigned to the first fluorescence lifetime, and wherein the second measure of fluorescence intensity comprises a second photon count of the second photons assigned to the second fluorescence lifetime.
7. The method of any preceding item, wherein determining each of the first measure and the second measure is based on determining an array of photon counts, each of a plurality of elements of the array comprising a value weighted according to a photon count corresponding to a respective one of a plurality of fluorescence lifetime values.
8. The method of any preceding item, further comprising comparing the ratio to a predetermined threshold ratio indicative of or aiding in diagnosis of a disease condition.
9. The method of any preceding item, further comprising assigning a probability of a disease condition based on the ratio.
10. The method of any preceding item, further comprising assigning a value corresponding to extent of progression of a disease condition based on the ratio.
11. The method of any preceding item, further comprising assigning a value corresponding to extent of progress of treatment of a disease condition based on the ratio.
12. The method of any preceding item, wherein at least one of the first fluorescence lifetime and the second fluorescence lifetime comprises a fluorescence lifetime of a signal indicative of a disease condition manifested at least in part in the ocular tissue, and wherein the disease condition comprises at least one of: an ocular disease; an amyloidogenic disorder and a pre-morbid neurodegenerative state.
13. The method of item 12, wherein the disease condition comprises Alzheimer's Disease.
14. The method of any preceding item, comprising determining the ratio at each of a plurality of times for a single individual's eyes, and determining an average ratio for the single individual based on the ratio at the plurality of times.
15. The method of any preceding item, comprising determining at least one of the first measure and the second measure based on at least one of a pixel weighted photon count over the area of the ocular tissue and an average photon count over the area of the ocular tissue.
16. The method of any preceding item, wherein the first measure comprises a first peak value of fluorescence intensity of the first photons assigned to the first fluorescence lifetime, and wherein the second measure comprises a second peak value of fluorescence intensity of the second photons assigned to the second fluorescence lifetime.
17. The method of any preceding item, wherein the first measure comprises a first value corresponding to the number or frequency of photons having a fluorescence lifetime within a first lifetime interval (dt₁) of the first fluorescence lifetime, and wherein the second measure comprises a second value corresponding to the number or frequency of photons having a fluorescence lifetime within a second lifetime interval (dt₂) of the second fluorescence lifetime.
18. The method of any preceding item, comprising illuminating the ocular tissue with a light source, thereby inducing emission of a plurality of photons comprising the first photons and the second photons.
19. The method of item 18,
   wherein the light source has at least one of a wavelength property, a polarization property or a combination thereof, each appropriate to produce fluorescence in the ocular tissue;
   the method further comprising receiving light including fluorescence produced as a result of the illuminating the eye, the light including the first photons and the second photons; and
   determining the first fluorescence lifetime for the first photons and the second fluorescence lifetime for the second photons based on the received light.
20. The method of item 19, further comprising performing a time correlation single photon count based on received electrical signals indicative of photon counts of the fluorescence produced as a result of illuminating the eye.
21. The method of any of items 18 through 20, wherein the light source comprises a pulsed light source.
22. The method of item 21, wherein the pulsed light source comprises a femto-second to nano-second pulsed light source.
23. The method of any preceding item, comprising illuminating the ocular tissue with multiple wavelengths of light in a single measurement.
24. The method of any preceding item, comprising:
   constructing a histogram of photon counts received as a function of time;
   fitting a multi-exponential decay curve to the histogram; and
   retrieving at least the first fluorescence lifetime and the second fluorescence lifetime from time decay rates of first and second respective component exponential decay curves of the multi-exponential decay curve.
25. A device configured to implement any of the methods of the preceding items.
26. A non-transient computer-readable storage medium having computer-readable code stored thereon, which, when executed by a computer processor, causes the computer processor to image ocular tissue, by causing the processor to implement any of the methods of the preceding items.

### EXAMPLES

A clinical trial was performed to evaluate the performance of the system in discriminating between a healthy volunteer (HV, N=20) group and patients diagnosed with Alzheimer's disease (AD, N=20).

Fluorescent Ligand, Aftobetin (compound #11), with an affinity upon binding to beta amyloid aggregates to fluoresce, was used as an exogenous ligand. The optical scanner device itself comprises of a pico-second pulsed laser (Becker & Hickl, Berlin) with a peak wavelength at 470 nm, pulse width 200 psec, 50 MHz repetition rate, and average output power of 10 uWatts. Fluorescence from excited molecules is collected in epi-fluorescence configuration, filtered with dichroic mirrors (Semrock Inc.) and an additional bandpass filter (centered at 585 nm) to reject remaining scattered laser light, and passed through an aperture to enable confocal detection. The detector is a single photon avalanche diode (MPD, Bolzano, Italy) with 50 ps FWHM timing resolution and efficiency of 50% at 550 nm.

All subjects were dosed with three doses of Fluorescent Ligand applied to the test eye two hours (+/−30 min) apart in the afternoon. A measurement session was conducted with the system the next morning, at 18 hrs. (+/−2) after the first dose.

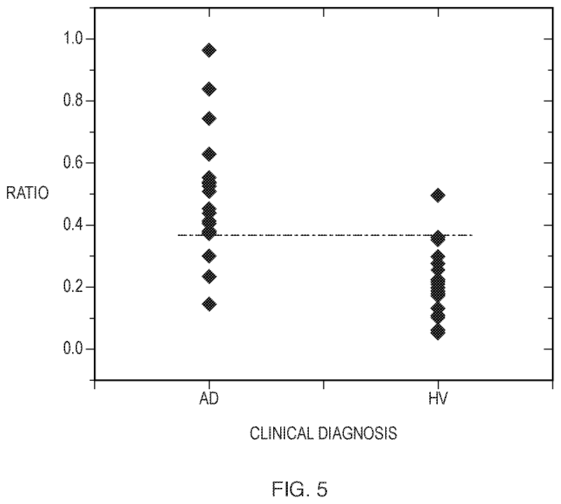

FIG. 5 shows the results (ratios-signal/background) obtained of the two groups, a threshold ratio around 0.37 can discriminate between the groups. Statistical analysis reveals a sensitivity of 85% and specificity of 95% (see Table 1).

TABLE 1
Descriptive Statistics by Clinical Diagnosis

| | HV | AD | |
|---|---|---|---|
| | N = 20 | N= 20 | Test Statistic |
| Sapphire II | | | χ₁² = 25.86, P < 0.001 |
| Negative | 95% 19/20 | 15% 3/20 | |
| Positive | 5% 1/20 | 85% 17/20 | |

Embodiments according to the present invention may make use of devices, techniques, fluorophore compounds and all other features taught in U.S. Patent Application Publication No. 2013/0135580 A1, the entire teachings of which application are hereby incorporated herein by reference. In particular, normalization methods, devices and computer-readable media according to embodiments of the present invention may be used in combination with the features taught in 2013/0135580 A1, for example in order to normalize fluorescent measurements obtained using the features taught in 2013/0135580 A1.

Portions of the above-described embodiments of the present invention can be implemented using one or more computer systems. For example, the embodiments may be implemented using hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, a tablet computer, a single circuit board computer or a system on a chip. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, touch screens and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, at least a portion of the invention may be embodied as a computer readable medium (or multiple computer readable media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement at least a portion of the various embodiments of the invention discussed above. The computer readable medium or media can be transportable, such that the program or programs stored thereon can be loaded onto one or more different computers or other processors to implement various aspects of the present invention as discussed above.

In this respect, it should be appreciated that one implementation of at least a portion of the above-described embodiments comprises at least one computer-readable medium encoded with a computer program (e.g., a plurality of instructions), which, when executed on a processor, performs some or all of the above-discussed functions of these embodiments. As used herein, the term “computer-readable medium” encompasses only a computer-readable medium that can be considered to be a machine or a manufacture (i.e., article of manufacture). A computer-readable medium may be, for example, a tangible medium on which computer-readable information may be encoded or stored, a storage medium on which computer-readable information may be encoded or stored, and/or a non-transitory medium on which computer-readable information may be encoded or stored. Other non-exhaustive examples of computer-readable media include a computer memory (e.g., a ROM, a RAM, a flash memory, or other type of computer memory), a magnetic disc or tape, an optical disc, and/or other types of computer-readable media that can be considered to be a machine or a manufacture.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method for measuring the amount of a fluorophore in ocular tissue, the method comprising the following steps:
a) contacting the ocular tissue with a first fluorophore that specifically binds to a protein;
b) illuminating the ocular tissue with a light source suitable to elicit fluorescence of the first fluorophore and suitable to elicit fluorescence of a second fluorophore, which is used as a reference;
c) determining a first light signal intensity for a selected lifetime value (τ1) or a lifetime interval (dt1) of the fluorescence emitted by the first fluorophore and a second light signal intensity for a selected lifetime value (τ2) or a lifetime interval (dt2) of the second fluorophore, wherein the first and second light signals are derived from the same region in the eye;
d) determining a ratio (r) of the first signal intensity to the second signal intensity, and
e) using the ratio (r) of the first to the second signal intensity for normalization of the determined light signal intensities.
